(19) United States
(12) Patent Application Publication
(10) Pub. No.: US 2021/0128554 A1
(43) Pub. Date: May 6, 2021

(54) ANTI-FLAVIVIRIDAE ACTIVITY OF ANTI-RETROVIRAL NON-NUCLEOSIDE (NNRTIS) AND NUCLEOSIDE REVERSE TRANSCRIPTASE INHIBITORS (NNRTIS)

(71) Applicant: Temple University - of the Commonwealth System of Higher Education, Philadelphia, PA (US)

(72) Inventor: Kamel Khalili, Bala Cynwyd, PA (US)

(21) Appl. No.: 16/472,462

(22) PCT Filed: Dec. 22, 2017
(86) PCT No.: PCT/US17/68132

§ 371 Date: Jun. 21, 2019

Related U.S. Application Data
(60) Provisional application No. 62/467,983, filed on Mar. 7, 2017.
(60) Provisional application No. 62/438,694, filed on Dec. 23, 2016.

(51) Int. Cl. A61K 31/505; A61P 31/14; A61K 45/06

(52) U.S. Cl. CPC A61K 31/505; A61K 45/06; A61P 31/14

(57) ABSTRACT

Compositions of non-nucleoside reverse transcriptase inhibitor (NNRTI) and/or a nucleoside reverse transcriptase inhibitor (NRTI) or combinations thereof, in the prevention and treatment of Flaviviridae infections, e.g. Zika virus.

### CROSS-REFERENCE TO RELATED APPLICATIONS

This application claims priority under 35 U.S.C. § 119 to U.S. Provisional Patent Application No. 62/438,694 filed Dec. 23, 2016 and U.S. Provisional Patent Application No. 62/467,983 filed Mar. 7, 2017, the entire contents of each of which is hereby expressly incorporated by reference herein.

### FIELD OF THE INVENTION

The present invention relates to compositions that inhibit replication of Flaviviruses, for example, Zika virus. In particular, the compositions comprise a non-nucleoside reverse transcriptase inhibitor (NNRTI) and/or a nucleoside reverse transcriptase inhibitor (NRTI) or combinations thereof. Such compositions can be administered to a subject having or at risk for contracting, for example, a Zika virus infection.

### BACKGROUND

Zika virus is an emerging virus with important public health consequences. Zika virus disease is caused by the Zika virus, which is spread to people primarily through the bite of an infected mosquito (Aedes aegypti and Aedes albopictus). Many people infected with Zika will have no symptoms or mild symptoms that last several days to a week. However, Zika infection during pregnancy can cause a serious birth defect called microcephaly and other severe fetal brain defects. Guillain-Barre syndrome (GBS), an uncommon sickness of the nervous system, is also very likely triggered by Zika in a small number of cases. Zika virus is an arbovirus (arthropod-borne virus) and a member of the family Flaviviridae, genus Flaviviridae. Zika virions are enveloped and icosahedral, and contain a nonsegmented, single-stranded, positive-sense RNA genome, which encodes 3 structural and 7 nonstructural proteins that are expressed as a single polyprotein that undergoes cleavage. Zika genomic RNA replicates in the cytoplasm of infected host cells. Zika virus was first detected in 1947 in the blood of a febrile monkey in Uganda's Zika Forest and in crushed suspensions of the Aedes mosquito, which is one of the vectors for Zika virus. The virus remained obscure, with a few human cases confined to Africa and Asia. There are two lineages of the Zika virus, African and Asian, with the Asian strain causing outbreaks in Micronesia in 2007 and French Polynesia in 2013-2014. From here, the virus spread to Brazil with the first report of autochthonous Zika transmission in the Americas in March 2015. The rapid advance of the virus in the Americas and its likely association with microcephaly and Guillain-Barré syndrome make Zika an urgent public health concern.

### SUMMARY

Embodiments of the invention are directed to compositions for inhibiting Flaviviridae replication and infection, in vitro or in vivo. Methods of treatment or prevention of an infection comprises the use of the compositions.

Other aspects are described infra.

### BRIEF DESCRIPTION OF THE DRAWINGS

FIGS. 1A, 1B show that Zika virus propagation is suppressed by Rilpivirine (TmC278) in primary human fetal and adult astrocytes: Primary human fetal and adult astrocytes were plated in 12 well tissue culture dishes and infected with ZIKV at 0.5 MOI. At 24, 48, and 72 hrs post-infections, cell were treated with increasing concentrations of anti-retroviral nucleoside analogs (DDI, ddC, AZT, FTC, ABA, LAM) and non-nucleoside reverse transcriptase inhibitors (Nevi and TmC278). Culture media were collected at 96 hrs post-infections and subjected to Real-time Q-RT-PCR for the detection and quantification of ZIKV copy numbers released by infected cells. DDI: 2′3′-Dideoxyinosine, ddC: 2′3′-Dideoxycytidine, AZT: Zidovudine, FTC: Emtricitabine, TmC278: Rilpivirine, Nevi: Nevirapine, ABA: Abacavir, LAM: Lamivudine.

FIG. 2 shows that Zika virus RNA replication is suppressed by Rilpivirine (TmC278) in primary human adult astrocytes: Primary human adult astrocytes were infected with ZIKV (0.5 MOI). At 24, 48, and 72 hrs post-infections, cells were treated with anti-retroviral nucleoside analogs (Aba, Lam) and non-nucleoside (TmC278) reverse transcriptase inhibitors. Cellular RNA was extracted at 96 hrs post-infections and subjected to Real-time Q-RT-PCR for the detection and quantification of ZIKV RNA copies in infected cells. TmC278: Rilpivirine, Aba: Abacavir, Lam: Lamivudine.

FIG. 3 shows that Zika virus gene expression is suppressed by Rilpivirine (TmC278) in primary human adult astrocytes: Primary human adult astrocytes were infected with ZIKV (0.5 MOI). At 24, 48, and 72 hrs post-infections, cells were treated with anti-retroviral nucleoside analogs (Aba, Lam) and non-nucleoside (TmC278) reverse transcriptase inhibitors. Cellular RNA was extracted at 96 hrs post-infections and subjected to semi quantitative RT-PCR for the amplification of ZIKV NS1, NS2A, and NS4A genes. PCR products were separated on a 1% agarose gel and stained with ethidium bromide. Actin was also amplified from same samples as the internal control. TmC278: Rilpivirine, Aba: Abacavir, Lam: Lamivudine.

FIG. 4 shows that Zika virus protein expression is suppressed by Rilpivirine (TmC278) in primary human adult astrocytes: Primary human adult astrocytes were infected with ZIKV (0.5 MOI). At 24, 48, and 72 hrs post-infections, cells were treated with anti-retroviral nucleoside analogs (Aba, Lam) and non-nucleoside (TmC) reverse transcriptase inhibitors. Whole cell protein lysates were extracted at 96 hrs post-infections and subjected to Western blotting for the detection of ZIKV-capsid and NS3 protein expression by utilizing specific antibodies. GAPDH was also probed in same membranes as the loading control. TmC278: Rilpivirine, Aba: Abacavir, Lam: Lamivudine.

FIGS. 5A, 5B are graphs showing the productive ZIKV (ATCC, VR-1843) replication in astrocytic cells is higher than microglia and hNPCs. FIG. 5A shows the real-time RT-PCR analysis of ZIKV copy numbers in supernatants of primary human fetal astrocytes (PHFA), primary human fetal microglia (PHFM), human glioblastoma cell line (U87MG), and hNPCs infected with 0.1 MOI ZIKV are performed at 4 dpi and represented as bar graph from three independent experiments. FIG. 5B shows the real-time RT-PCR analysis of ZIKV copy numbers in supernatants and cellular RNA samples of primary human fetal astrocytes (PHFA) infected with 0.5 MOI ZIKV are performed at 0, 1, 2, 3, and 4 dpi and represented over-time from three independent experiments.

FIG. 6 is a graph showing that Zika virus replication is suppressed by antiretroviral HIV-1 non-nucleoside reverse transcriptase inhibitors (NNRTIs) in primary astrocytes: Primary human fetal astrocytes were plated in 6 well tissue culture dishes and infected with ZIKV at 0.1 MOI. At 24, 48, and 72 hrs post-infections, cells were treated with increasing concentrations of anti-retroviral non-nucleoside reverse transcriptase inhibitors Rilpivirine, Efavirenz, and Etravirine at 5, 10, and 25 μM concentrations. Culture media of the infected cells were collected at 96 hrs post-infections and subjected to Real-time Q-RT-PCR for the detection and quantification of ZIKV copy numbers released by infected cells.

FIGS. 7A-7D are graphs showing that rilpivirine inhibits ZIKV in IFNR^{-/-} mice.

### DETAILED DESCRIPTION

Embodiments are directed to compositions that suppress replication of Flaviviridae, e.g. Zika virus. Methods include, the prevention and treatment of subjects at risk of being infected, e.g. living or travelling to a location with, e.g. Zika virus infections.

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used.

It is also to be understood that the terminology used herein is for the purpose of describing particular embodiments only, and is not intended to be limiting.

The articles “a” and “an” are used herein to refer to one or to more than one (i.e., to at least one) of the grammatical object of the article. By way of example, “an element” means one element or more than one element. Thus, recitation of “a cell”, for example, includes a plurality of the cells of the same type. Furthermore, to the extent that the terms “including”, “includes”, “having”, “has”, “with”, or variants thereof are used in either the detailed description and/or the claims, such terms are intended to be inclusive in a manner similar to the term “comprising.”

“About” as used herein when referring to a measurable value such as an amount, a temporal duration, and the like, is meant to encompass variations of +/−20%, +/−10%, +/−5%, +/−1%, or +/−0.1% from the specified value, as such variations are appropriate to perform the disclosed methods. Alternatively, particularly with respect to biological systems or processes, the term can mean within an order of magnitude within 5-fold, and also within 2-fold, of a value. Where particular values are described in the application and claims, unless otherwise stated the term “about” meaning within an acceptable error range for the particular value should be assumed.

The term “anti-viral agent” as used herein, refers to any molecule that is used for the treatment of a virus and include agents which alleviate any symptoms associated with the virus, for example, anti-pyretic agents, anti-inflammatory agents, chemotherapeutic agents, and the like. An antiviral agent includes, without limitation: antibodies, aptamers, adjuvants, anti-sense oligonucleotides, chemokines, cytokines, immune stimulating agents, immune modulating agents, B-cell modulators, T-cell modulators, NK cell modulators, antigen presenting cell modulators, enzymes, siRNA's, ribavirin, protease inhibitors, helicase inhibitors, polymerase inhibitors, helicase inhibitors, neuraminidase inhibitors, nucleoside reverse transcriptase inhibitors, non-nucleoside reverse transcriptase inhibitors, purine nucleosides, chemokine receptor antagonists, interleukins, or combinations thereof.

The term “antibody” as used herein comprises one or more virus specific binding domains which bind to and aid in the immune mediated-destruction and clearance of the virus, e.g. Zika virus. The antibody or fragments thereof, comprise IgA, IgM, IgG, IgE, IgD or combinations thereof.

The terms, “compound” and “compounds” as used herein refers to non-nucleoside reverse transcriptase inhibitors (NNRTIs), nucleoside reverse transcriptase inhibitors (NRTIs), analogs, variants etc.

As used herein, the terms “comprising,” “comprise” or “comprised,” and variations thereof, in reference to defined or described elements of an item, composition, apparatus, method, process, system, etc. are meant to be inclusive or open ended, permitting additional elements, thereby indicating that the defined or described item, composition, apparatus, method, process, system, etc. includes those specified elements—or, as appropriate, equivalents thereof—and that other elements can be included and still fall within the scope/definition of the defined item, composition, apparatus, method, process, system, etc.

“Optional” or “optionally” means that the subsequently described event or circumstance can or cannot occur, and that the description includes instances where the event or circumstance occurs and instances where it does not.

As used in this specification and the appended claims, the term “or” is generally employed in its sense including “and/or” unless the content clearly dictates otherwise.

The terms “patient” or “individual” or “subject” are used interchangeably herein, and refers to a mammalian subject to be treated, with human patients being preferred. In some cases, the methods of the invention find use in experimental animals, in veterinary application, and in the development of animal models for disease, including, but not limited to, rodents including mice, rats, and hamsters, and primates.

To “treat” a disease as the term is used herein, means to reduce the frequency or severity of at least one sign or symptom of a disease or disorder experienced by a subject. Treatment of a disease or disorders includes the eradication of a virus.

“Treatment” is an intervention performed with the intention of preventing the development or altering the pathology or symptoms of a disorder. Accordingly, “treatment” refers to both therapeutic treatment and prophylactic or preventative measures. “Treatment” may also be specified as palliative care. Those in need of treatment include those already with the disorder as well as those in which the disorder is to be prevented. Accordingly, “treating” or “treatment” of a state, disorder or condition includes: (1) eradicating the virus; (2) preventing or delaying the appearance of clinical symptoms of the state, disorder or condition developing in a human or other mammal that may be afflicted with or predisposed to the state, disorder or condition but does not yet experience or display clinical or subclinical symptoms of the state, disorder or condition; (3) inhibiting the state, disorder or condition, i.e., arresting, reducing or delaying the development of the disease or a relapse thereof (in case of maintenance treatment) or at least one clinical or subclinical symptom thereof; or (4) relieving the disease, i.e., causing regression of the state, disorder or condition or at least one of its clinical or subclinical symptoms. The benefit to an individual to be treated is either statistically significant or at least perceptible to the patient or to the physician.

As defined herein, a “therapeutically effective” amount of a compound or agent (i.e., an effective dosage) means an amount sufficient to produce a therapeutically (e.g., clinically) desirable result. The compositions can be administered from one or more times per day to one or more times per week; including once every other day. The skilled artisan will appreciate that certain factors can influence the dosage and timing required to effectively treat a subject, including but not limited to the severity of the disease or disorder, previous treatments, the general health and/or age of the subject, and other diseases present. Moreover, treatment of a subject with a therapeutically effective amount of the compounds of the invention can include a single treatment or a series of treatments.

As defined herein, an “effective” amount of a compound or agent (i.e., an effective dosage) means an amount sufficient to produce a (e.g., clinically) desirable result.

As used herein, a “pharmaceutically acceptable” component/carrier etc. is one that is suitable for use with humans and/or animals without undue adverse side effects (such as toxicity, irritation, and allergic response) commensurate with a reasonable benefit/risk ratio.

### Compositions for Inhibiting Replication of Flaviviridae

No clinically approved therapy is currently available for the treatment of Zika or indeed any other Flaviviridae infection (Lim et al., 2013, Antiviral Res 100: 500-519). Over the past decade, significant effort has been made towards dengue drug discovery. Due to the similarity between Zika virus and dengue virus, it is possible that knowledge from dengue drug discovery could be applied to Zika virus. Several approaches are possible, e.g., high-throughput screening using virus replication assays or viral enzyme assays, structure-based in silico docking and rational design strategies and repurposing hepatitis C virus inhibitors for Zika. The development of antivirals should focus on distinctive features of Zika molecular biology that can be exploited. For example, Zika NS3 protein has a protease activity that is necessary for the viral life cycle and this may be a viable target for small molecule antiviral inhibitors. In this regard, the inhibitors of the NS3/4A protease of Hepatitis C, telaprevir and boceprevir, revolutionize the management of hepatitis C genotype 1 patients (Vermehren and Sarrazin, 2011, Eur J Med Res 16: 303-314). NS3 also has a 5′-RNA triphosphatase activity required for 5′-RNA cap formation and NS5 contains a C-terminal RNA-dependent RNA polymerase (RdRp) activity as described above and these are also potential targets for the development of small molecule antiviral inhibitors (Lim et al, 2015, Antiviral Res 100: 500-519; Luo et al, 2015, Antiviral Res 118: 148-158). Finally, the advent of methodologies such as the CRISPR/Cas9 system that are specifically able to target nucleotide sequences within viral genomes has provided an effective, specific, and versatile weapon against human DNA viruses (White et al, 2015, Discov Med 19: 255-262).

Accordingly, compositions embodied herein are directed to the inhibition of Flaviviridae virus replication.

In certain embodiments, a composition comprises a therapeutically effective amount of a non-nucleoside reverse transcriptase inhibitor (NNRTI) and/or a nucleoside reverse transcriptase inhibitor (NRTI), analogs, variants or combinations thereof. In certain embodiments, an NNRTI comprises: etravirine, efavirenz, nevirapine, rilpivirine, delavirdine, or nevirapine. In embodiments, an NRTI comprises: lamivudine, zidovudine, emtricitabine, abacavir, zalcitabine, dideoxycytidine, azidothymidine, tenofovir disoproxil fumarate, didanosine (ddI EC), dideoxyinosine, stavudine, abacavir sulfate or combinations thereof.

In certain embodiments, a composition comprises a therapeutically effective amount of at least one NNRTI or a combination of NNRTI's, analogs, variants or combinations thereof. In certain embodiments, the NNRTI is rilpivirine.

In certain embodiments, an NRTI comprises: lamivudine, zidovudine, emtricitabine, abacavir, zalcitabine, dideoxycytidine, azidothymidine, tenofovir disoproxil fumarate, didanosine (ddI EC), dideoxyinosine, stavudine, abacavir sulfate or combinations thereof. In certain embodiments, the composition comprises a therapeutically effective amount of at least one or a combination of NRTI's, analogs, variants or combinations thereof.

In other embodiments, a composition comprises a therapeutically effective amount of a combination of at least one or more NNRTI's and a therapeutically effective amount of at least one or more NRTI's. In certain embodiments, the at least one NNRTI is rilpivirine.

In certain embodiments, the composition further comprises at least one or more protease inhibitors. In certain embodiments, a protease inhibitor comprises: amprenavir, tipranavir, indinavir, saquinavir mesylate, lopinavir and ritonavir (LPV/RTV), Fosamprenavir Calcium (FOS-APV), ritonavir, darunavir, atazanavir sulfate, nelfinavir mesylate or combinations thereof.

Flaviviridae viruses included within the scope of this invention are discussed generally in Fields Virology, Editors: Fields, N., Knipe, D. M. and Howley, P. M.; Lippincott-Raven Publishers, Philadelphia, Pa.; Chapter 31 (1996). Specific Flaviviridae include, without limitation: Absettarov; Alfuy; Apoi; Aroa; Bagaza; Banzi; Bououi; Bussuquara; Cacipacore; Carey Island; Dakar bat; Dengue viruses 1, 2, 3 and 4; Edge Hill; Entebbe bat; Gadgets Gully; Hanzalova; Hypr; Ilheus; Israel turkey meningoencephalitis; Japanese encephalitis; Jugra; Jutiapa; Kadam; Karshi; Kedougou; Kokoera; Koutango; Kumlinge; Kunjin; Kyasanur Forest virus; Langat; Louping ill; Meaban; Modoc; Montana myotis leukoencephalitis; Murray valley encephalitis; Naranjal; Negishi; Ntaya; Omsk hemorrhagic fever; Phnom-Penh bat; Powassan; Rio Bravo; Rocio; Royal Farm; Russian spring-summer encephalitis; Saboya; St. Louis encephalitis; Sal Vieja; San Perlita; Saumarez Reef Sepik; Sokuluk; Spondweni; Stratford; Temusu; Tyuleniy; Uganda S, Usutu, Wesselsbron; West Nile; Yaounde; Yellow fever; and Zika.

In certain embodiments, the Flaviviridae comprise: Dengue Fever Virus, West Nile Fever Virus, Yellow Fever Virus, St. Louis Encephalitis Virus, Japanese Encephalitis Virus, Murray Valley Encephalitis Virus, Tick-borne Encephalitis Virus, Kunjin Encephalitis Virus, Rocio Encephalitis Virus, Russian Spring Summer Encephalitis Virus, Negishi Virus, Kyasanur Forest Virus, Omsk Hemorrhagic Fever Virus, Powassan Virus, Louping III Virus, Rio Bravo Virus, Tyuleniy Virus, Ntaya Virus, Modoc Virus, Alkhurma Hemorrhagic Fever Virus, Zika virus.

In one embodiment, the Flaviviridae is Zika virus.

In addition, one or more agents which alleviate any other symptoms that may be associated with the virus infection, e.g. fever, chills, headaches, secondary infections, can be administered in concert with, or as part of the pharmaceutical composition or at separate times. These agents comprise, without limitation, an anti-pyretic agent, anti-inflammatory agent, chemotherapeutic agent, or combinations thereof.

In certain embodiments, a composition comprises a therapeutically effective amount of a combination of at least one or more NNRTI's, analogs, variants or combinations thereof, and/or a therapeutically effective amount of at least one or more NRTI's, analogs, variants or combinations thereof are administered with one or more therapeutic agents comprising anti-viral agents and/or agents which alleviate any disorder or symptoms associated with a virus or secondary infections. Examples of disorders include: neurological disorders, tumors, inflammation etc.

In certain embodiments, the compositions further comprise one or more agents which alleviate any other symptoms that may be associated with the virus infection, e.g. fever, chills, headaches, secondary infections, can be administered in concert with, or as part of the pharmaceutical composition or at separate times. These agents comprise, without limitation, anti-pyretic agents, anti-inflammatory agents, chemotherapeutic agents, antibiotics, anti-fungal, chemotherapeutic agents, interferons, cytokines, monokines, antibodies or combinations thereof.

In certain embodiments, the anti-viral agent comprises therapeutically effective amounts of: antibodies, aptamers, adjuvants, anti-sense oligonucleotides, chemokines, cytokines, immune stimulating agents, immune modulating molecules, B-cell modulators, T-cell modulators, NK cell modulators, antigen presenting cell modulators, enzymes, siRNA's, interferon, ribavirin, protease inhibitors, anti-sense oligonucleotides, helicase inhibitors, polymerase inhibitors, helicase inhibitors, neuraminidase inhibitors, nucleoside reverse transcriptase inhibitors, non-nucleoside reverse transcriptase inhibitors, purine nucleosides, chemokine receptor antagonists, interleukins, vaccines or combinations thereof.

The immune-modulating molecules comprise, but are not limited to cytokines, lymphokines, T cell co-stimulatory ligands, etc. An immune-modulating molecule positively and/or negatively influences the humoral and/or cellular immune system, particularly its cellular and/or non-cellular components, its functions, and/or its interactions with other physiological systems. The immune-modulating molecule may be selected from the group comprising cytokines, chemokines, macrophage migration inhibitory factor (MIF; as described, inter alia, in Bernhagen (1998), Mol Med 76(3-4); 151-61 or Metz (1997), Adv Immunol 66, 197-223), T-cell receptors or soluble MHC molecules. Such immune-modulating effector molecules are well known in the art and are described, inter alia, in Paul, “Fundamental immunology”, Raven Press, New York (1989). In particular, known cytokines and chemokines are described in Meager, “The Molecular Biology of Cytokines” (1998), John Wiley & Sons, Ltd., Chichester, West Sussex, England; (Bacon (1998). Cytokine Growth Factor Rev 9(2):167-73; Oppenheim (1997). Clin Cancer Res 12, 2682-6; Taub, (1994) Ther. Immunol. 1(4), 229-46 or Michiel, (1992). Semin Cancer Biol 3(1), 3-15).

Immune cell activity that may be measured include, but is not limited to, (1) cell proliferation by measuring the DNA replication; (2) enhanced cytokine production, including specific measurements for cytokines, such as IFN-γ, GM-CSF, or TNF-α; (3) cell mediated target killing or lysis; (4) cell differentiation; (5) immunoglobulin production; (6) phenotypic changes; (7) production of chemotactic factors or chemotaxis, meaning the ability to respond to a chemotactin with chemotaxis; (8) immunosuppression, by inhibition of the activity of some other immune cell type; and, (9) apoptosis, which refers to fragmentation of activated immune cells under certain circumstances, as an indication of abnormal activation.

Also of interest are enzymes present in the lytic package that cytotoxic T lymphocytes or LAK cells deliver to their targets. Perforin, a pore-forming protein, and Fas ligand are major cytolytic molecules in these cells (Brandau et al., Clin. Cancer Res. 6:3729, 2000; Cruz et al., Br. J. Cancer 81:881, 1999). CTLs also express a family of at least 11 serine proteases termed granzymes, which have four primary substrate specificities (Kam et al., Biochim. Biophys. Acta 1477:307, 2000). Low concentrations of streptolysin O and pneumolysin facilitate granzyme B-dependent apoptosis (Browne et al., Mol. Cell Biol. 19:8604, 1999).

Other suitable effectors encode polypeptides having activity that is not itself toxic to a cell, but renders the cell sensitive to an otherwise nontoxic compound—either by metabolically altering the cell, or by changing a non-toxic prodrug into a lethal drug. Exemplary is thymidine kinase (tk), such as may be derived from a herpes simplex virus, and catalytically equivalent variants. The HSV tk converts the anti-herpetic agent ganciclovir (GCV) to a toxic product that interferes with DNA replication in proliferating cells.

Methods of Prevention and/or Treatment of a Flaviviridae Infection.

In certain embodiments, a method of inhibiting a Flaviviridae virus replication in a cell, in vitro or in vivo comprises contacting a cell in vitro or administering to a subject, a therapeutically effective amount of a non-nucleoside reverse transcriptase inhibitor (NNRTI), analogs, variants or combinations thereof and/or a nucleoside reverse transcriptase inhibitor (NRTI), analogs, variants or combinations thereof.

In certain embodiments, a method of preventing and/or treating a subject at risk of contracting a Zika virus infection or infected with Zika virus, comprises administering to a subject, a therapeutically effective amount of a non-nucleoside reverse transcriptase inhibitor (NNRTI), analogs, variants or combinations thereof and/or a nucleoside reverse transcriptase inhibitor (NRTI), analogs, variants or combinations thereof, thereby preventing and/or treating a subject at risk of contracting a Zika virus infection or infected with Zika virus.

In certain embodiments, an NNRTI comprises: etravirine, efavirenz, nevirapine, rilpivirine, delavirdine, or nevirapine. In some embodiments, a therapeutically effective amount of at least one NNRTI or a combination of NNRTI's are administered to a subject. In certain embodiments the at least one NNRTI is rilpivirine. In certain embodiments, an NRTI comprises: lamivudine, zidovudine, emtricitabine, abacavir, zalcitabine, dideoxycytidine, azidothymidine, tenofovir disoproxil fumarate, didanosine (ddI EC), dideoxyinosine, stavudine, abacavir sulfate or combinations thereof. In some embodiments, a therapeutically effective amount of at least one or a combination of NRTI's are administered to a subject. In other embodiments, a combination of a therapeutically effective amount of at least one or more NNRTI's and a therapeutically effective amount of at least one or more NRTI's are administered to a subject. In certain embodiments, a therapeutically effective amount of at least one or a combination of two or more NRTI's are administered to a subject. In certain embodiments, a combination of a therapeutically effective amount of at least one or more NNRTI's and a therapeutically effective amount of at least one or more NRTI's are administered to a subject. In certain embodiments the at least one NNRTI is rilpivirine.

In certain embodiments, the method further comprises one or more protease inhibitors. In embodiments, a protease inhibitor comprises: amprenavir, tipranavir, indinavir, saquinavir mesylate, lopinavir and ritonavir (LPV/RTV), Fosamprenavir Calcium (FOS-APV), ritonavir, darunavir, atazanavir sulfate, nelfinavir mesylate or combinations thereof.

In embodiments, the therapeutically effective amount of at least one NNRTI, NRTI or combinations thereof are administered as a pharmaceutical composition. In other embodiments, a therapeutically effective amount of at least one NNRTI, NRTI or combinations thereof are comprised within a delivery vehicle or administered as part of the delivery vehicle.

### Delivery Vehicles and Pharmaceutical Compositions

Delivery vehicles as used herein, include any types of molecules for delivery of the compositions embodied herein, both for in vitro or in vivo delivery. Examples, include, without limitation: nanoparticles, colloidal compositions, lipids, liposomes, nanosomes, carbohydrates, organic or inorganic compositions and the like.

The compositions of the invention can be delivered to an appropriate cell of a subject. This can be achieved by, for example, the use of a polymeric, biodegradable microparticle or microcapsule delivery vehicle, sized to optimize phagocytosis by phagocytic cells such as macrophages. For example, PLGA (poly-lacto-co-glycolide) microparticles approximately 1-10 μm in diameter can be used. The polynucleotide is encapsulated in these microparticles, which are taken up by macrophages and gradually biodegraded within the cell, thereby releasing the composition(s). A second type of microparticle is intended not to be taken up directly by cells, but rather to serve primarily as a slow-release reservoir of the composition(s) that is taken up by cells only upon release from the micro-particle through biodegradation. These polymeric particles should therefore be large enough to preclude phagocytosis (i.e., larger than 5 μm and preferably larger than 20 μm). Another way to achieve uptake of the composition(s) is using liposomes, prepared by standard methods. The composition(s) can be incorporated alone into these delivery vehicles or co-incorporated with tissue-specific antibodies, for example antibodies that target cell types that are commonly infected for example, brain cells, neurons etc.

In some embodiments, the compositions of the invention can be formulated as a nanoparticle, for example, nanoparticles comprised of a core of high molecular weight linear polyethylenimine (LPEI) complexed with DNA and surrounded by a shell of polyethyleneglycol modified (PEGylated) low molecular weight LPEI.

Suitable pharmaceutical carriers, as well as pharmaceutical necessities for use in pharmaceutical formulations, are described in Remington's Pharmaceutical Sciences (E. W. Martin), a well-known reference text in this field, and in the USP/NF (United States Pharmacopeia and the National Formulary).

In some embodiments, the compositions may be formulated as a topical gel for blocking sexual transmission of, for example the Zika virus. The topical gel can be applied directly to the skin or mucous membranes of the male or female genital region prior to sexual activity. Alternatively, or in addition the topical gel can be applied to the surface or contained within a male or female condom or diaphragm.

In some embodiments, the compositions can be formulated as a nanoparticle encapsulating the compositions embodied herein.

Any of the pharmaceutical compositions of the invention can be formulated for use in the preparation of a medicament, and particular uses are indicated below in the context of treatment, e.g., the treatment of a subject having a Zika viral infection or at risk for contracting a Zika virus infection. When employed as pharmaceuticals, any of the composition(s) can be administered in the form of pharmaceutical compositions. These compositions can be prepared in a manner well known in the pharmaceutical art, and can be administered by a variety of routes, depending upon whether local or systemic treatment is desired and upon the area to be treated. Administration may be topical (including ophthalmic and to mucous membranes including intranasal, vaginal and rectal delivery), pulmonary (e.g., by inhalation or insufflation of powders or aerosols, including by nebulizer; intratracheal, intranasal, epidermal and transdermal), ocular, oral or parenteral. Methods for ocular delivery can include topical administration (eye drops), subconjunctival, periocular or intravitreal injection or introduction by balloon catheter or ophthalmic inserts surgically placed in the conjunctival sac. Parenteral administration includes intravenous, intraarterial, subcutaneous, intraperitoneal or intramuscular injection or infusion; or intracranial, e.g., intrathecal or intraventricular administration. Parenteral administration can be in the form of a single bolus dose, or may be, for example, by a continuous perfusion pump. Pharmaceutical compositions and formulations for topical administration may include transdermal patches, ointments, lotions, creams, gels, drops, suppositories, sprays, liquids, powders, and the like. Conventional pharmaceutical carriers, aqueous, powder or oily bases, thickeners and the like may be necessary or desirable.

The pharmaceutical compositions may contain, as the active ingredient, nucleic acids and vectors described herein in combination with one or more an antiviral agent, or combinations thereof in pharmaceutically acceptable carriers. In addition, one or more agents which alleviate any other symptoms that may be associated with the virus infection, e.g. fever, chills, headaches, secondary infections, can be administered in concert with, or as part of the pharmaceutical composition or at separate times. These agents comprise, without limitation, an anti-pyretic agent, anti-inflammatory agent, chemotherapeutic agent, antibiotics or combinations thereof.

In making the compositions of the invention, the active ingredient is typically mixed with an excipient, diluted by an excipient or enclosed within such a carrier in the form of, for example, a capsule, tablet, sachet, paper, or other container. When the excipient serves as a diluent, it can be a solid, semisolid, or liquid material (e.g., normal saline), which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), lotions, creams, ointments, gels, soft and hard gelatin capsules, suppositories, sterile injectable solutions, and sterile packaged powders. As is known in the art, the type of diluent can vary depending upon the intended route of administration. The resulting compositions can include additional agents, such as preservatives. In some embodiments, the carrier can be, or can include, a lipid-based or polymer-based colloid. In some embodiments, the carrier material can be a colloid formulated as a liposome, a hydrogel, a microparticle, a nanoparticle, or a block copolymer micelle. As noted, the carrier material can form a capsule, and that material may be a polymer-based colloid.

Any composition described herein can be administered to any part of the host's body for subsequent delivery to a target cell. A composition can be delivered to, without limitation, the brain, the cerebrospinal fluid, joints, nasal mucosa, blood, lungs, intestines, muscle tissues, skin, or the peritoneal cavity of a mammal. In terms of routes of delivery, a composition can be administered by intravenous, intracranial, intraperitoneal, intramuscular, subcutaneous, intramuscular, intrarectal, intravaginal, intrathecal, intratracheal, intradermal, or transdermal injection, by oral or nasal administration, or by gradual perfusion over time. In a further example, an aerosol preparation of a composition can be given to a host by inhalation.

The dosage required will depend on the route of administration, the nature of the formulation, the nature of the patient's illness, the patient's size, weight, surface area, age, and sex, other drugs being administered, and the judgment of the attending clinicians. Wide variations in the needed dosage are to be expected in view of the variety of cellular targets and the differing efficiencies of various routes of administration. Variations in these dosage levels can be adjusted using standard empirical routines for optimization, as is well understood in the art. Administrations can be single or multiple (e.g., 2- or 3-, 4-, 6-, 8-, 10-, 20-, 50-, 100-, 150-, or more fold). Encapsulation of the compounds in a suitable delivery vehicle (e.g., polymeric microparticles or implantable devices) may increase the efficiency of delivery.

The duration of treatment with any composition provided herein can be any length of time from as short as one day to as long as the life span of the host (e.g., many years). For example, a compound can be administered once a week (for, for example, 4 weeks to many months or years); once a month (for, for example, three to twelve months or for many years); or once a year for a period of 5 years, ten years, or longer. It is also noted that the frequency of treatment can be variable. For example, the present compounds can be administered once (or twice, three times, etc.) daily, weekly, monthly, or yearly.

An effective amount of any composition provided herein can be administered to an individual in need of treatment. An effective amount can be determined by assessing a patient's response after administration of a known amount of a particular composition. In addition, the level of toxicity, if any, can be determined by assessing a patient's clinical symptoms before and after administering a known amount of a particular composition. It is noted that the effective amount of a particular composition administered to a patient can be adjusted according to a desired outcome as well as the patient's response and level of toxicity. Significant toxicity can vary for each particular patient and depends on multiple factors including, without limitation, the patient's disease state, age, and tolerance to side effects.

Dosage, toxicity and therapeutic efficacy of such compositions can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀.

The data obtained from the cell culture assays and animal studies can be used in formulating a range of dosage for use in humans. The dosage of such compositions lies preferably within a range of circulating concentrations that include the ED₅₀ with little or no toxicity. The dosage may vary within this range depending upon the dosage form employed and the route of administration utilized. For any composition used in the method of the invention, the therapeutically effective dose can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

As described, a therapeutically effective amount of a composition (i.e., an effective dosage) means an amount sufficient to produce a therapeutically (e.g., clinically) desirable result. The compositions can be administered one from one or more times per day to one or more times per week; including once every other day. The skilled artisan will appreciate that certain factors can influence the dosage and timing required to effectively treat a subject, including but not limited to the severity of the disease or disorder, previous treatments, the general health and/or age of the subject, and other diseases present. Moreover, treatment of a subject with a therapeutically effective amount of the compositions of the invention can include a single treatment or a series of treatments.

Kits

The compositions described herein can be packaged in suitable containers labeled, for example, for use as a therapy to treat a subject having a Flaviviridae infection, for example, a Zika virus infection or a subject at risk of contracting for example, a Zika virus infection. The containers can include a composition comprising at least one NNRTI, e.g. Rilpivirine and one or more of a suitable stabilizer, carrier molecule, flavoring, and/or the like, as appropriate for the intended use. In other embodiments, the kit further comprises one or more anti-viral agents and/or therapeutic reagents that alleviate some of the symptoms or secondary bacterial infections that may be associated with a Flaviviridae infection. Accordingly, packaged products (e.g., sterile containers containing one or more of the compositions described herein and packaged for storage, shipment, or sale at concentrated or ready-to-use concentrations) and kits, including at least one composition of the invention, e.g., Rilpivirine and instructions for use, are also within the scope of the invention. A product can include a container (e.g., a vial, jar, bottle, bag, or the like) containing one or more compositions of the invention. In addition, an article of manufacture further may include, for example, packaging materials, instructions for use, syringes, delivery devices, buffers or other control reagents for treating or monitoring the condition for which prophylaxis or treatment is required.

The product may also include a legend (e.g., a printed label or insert or other medium describing the product's use (e.g., an audio- or videotape)). The legend can be associated with the container (e.g., affixed to the container) and can describe the manner in which the compositions therein should be administered (e.g., the frequency and route of administration), indications therefor, and other uses. The compositions can be ready for administration (e.g., present in dose-appropriate units), and may include one or more additional pharmaceutically acceptable adjuvants, carriers or other diluents and/or an additional therapeutic agent. Alternatively, the compositions can be provided in a concentrated form with a diluent and instructions for dilution.

While various embodiments of the present invention have been described above, it should be understood that they have been presented by way of example only, and not limitation. Numerous changes to the disclosed embodiments can be made in accordance with the disclosure herein without departing from the spirit or scope of the invention. Thus, the breadth and scope of the present invention should not be limited by any of the above described embodiments.

All documents mentioned herein are incorporated herein by reference. All publications and patent documents cited in this application are incorporated by reference for all purposes to the same extent as if each individual publication or patent document were so individually denoted. By their citation of various references in this document, applicants do not admit any particular reference is “prior art” to their invention.

### EXAMPLES

The present invention is further illustrated by the following specific examples. The examples are provided for illustration only and are not to be construed as limiting the scope or content of the invention in any way.

### Example 1: Reverse-Transcriptase Inhibitors (RTIs)

Reverse-transcriptase inhibitors (RTIs) are a class of antiretroviral drugs used to treat HIV infection or AIDS. RTIs inhibit activity of reverse transcriptase, a viral DNA polymerase that is required for replication of HIV and other retroviruses.

The question addressed herein, was whether this class of anti-retroviral drugs could have any impact on ZIKV replication and gene expression in human astrocytes.

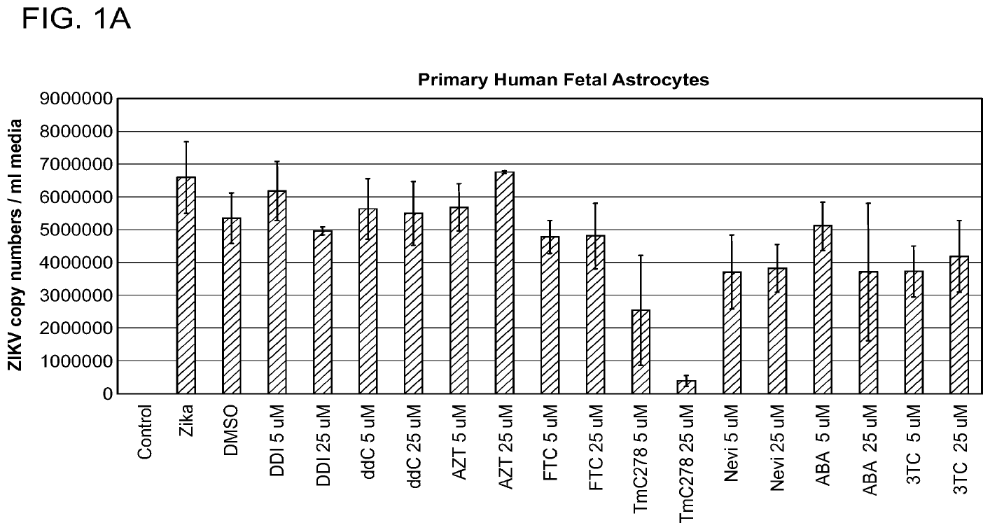

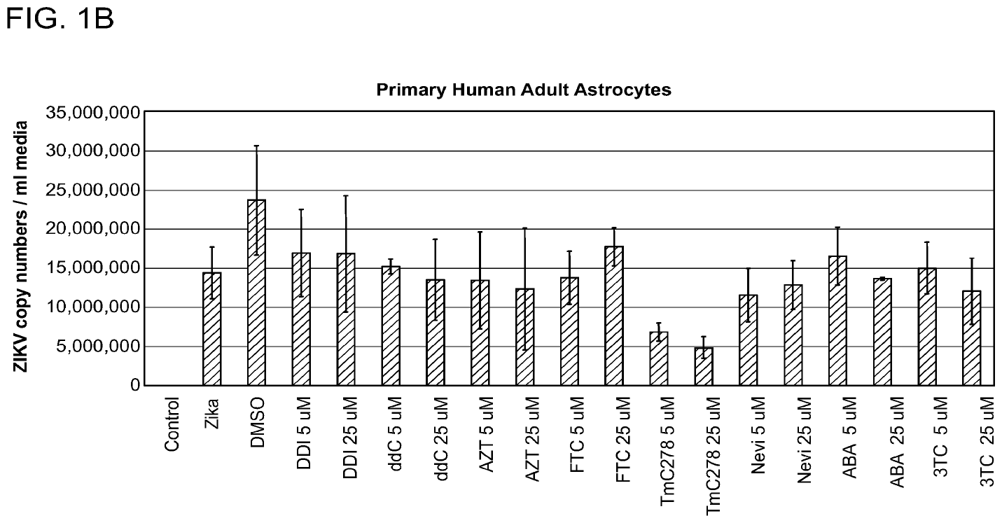

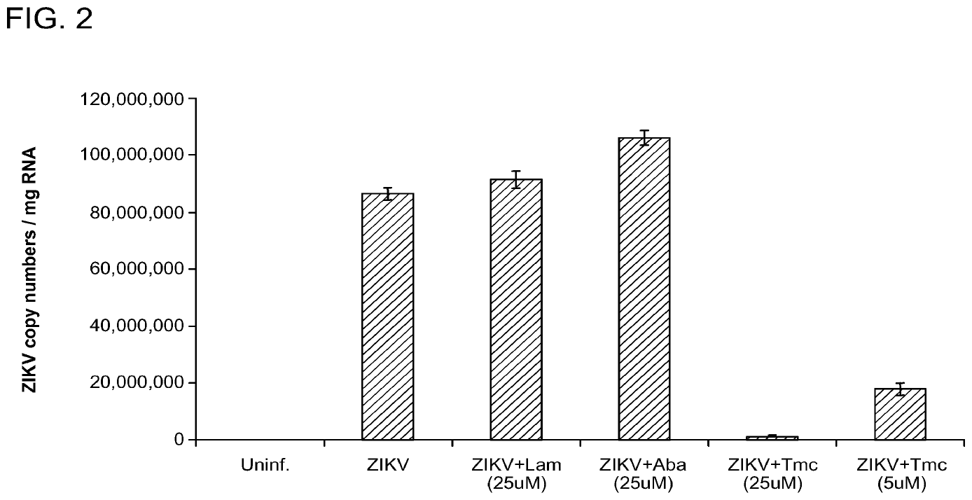

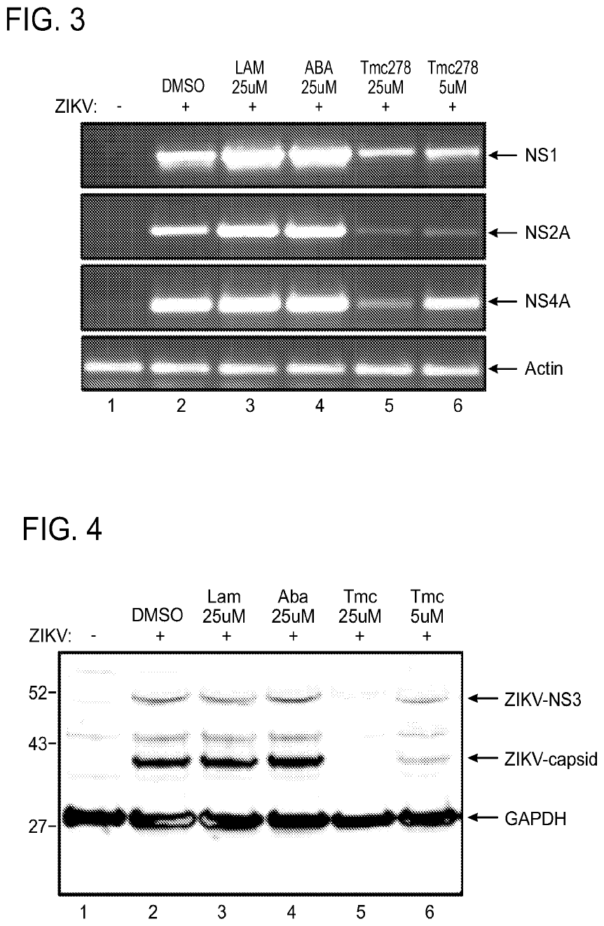

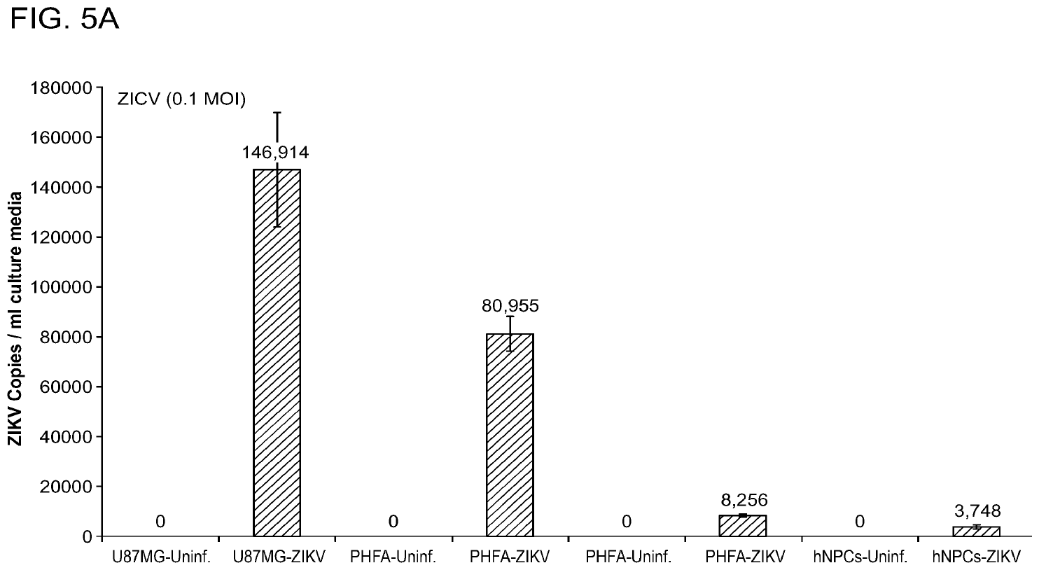

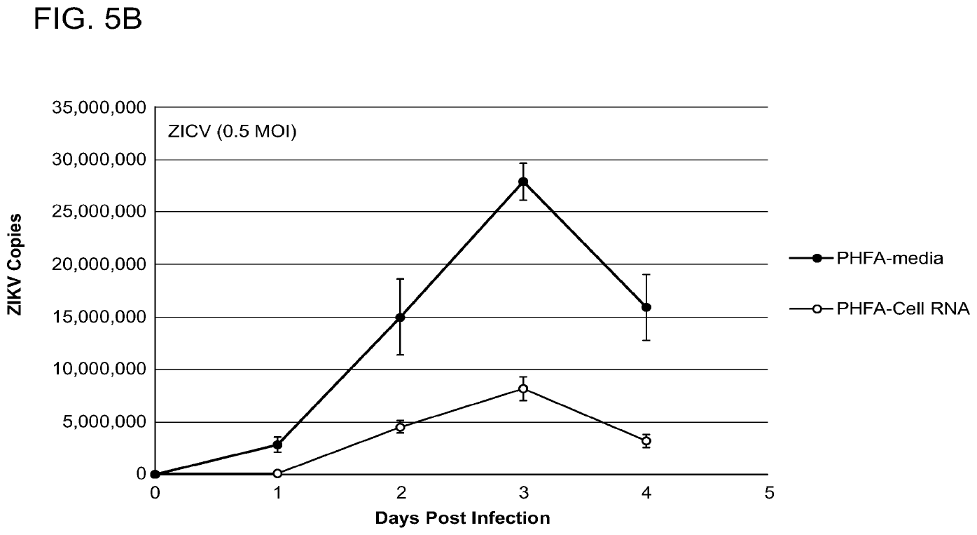

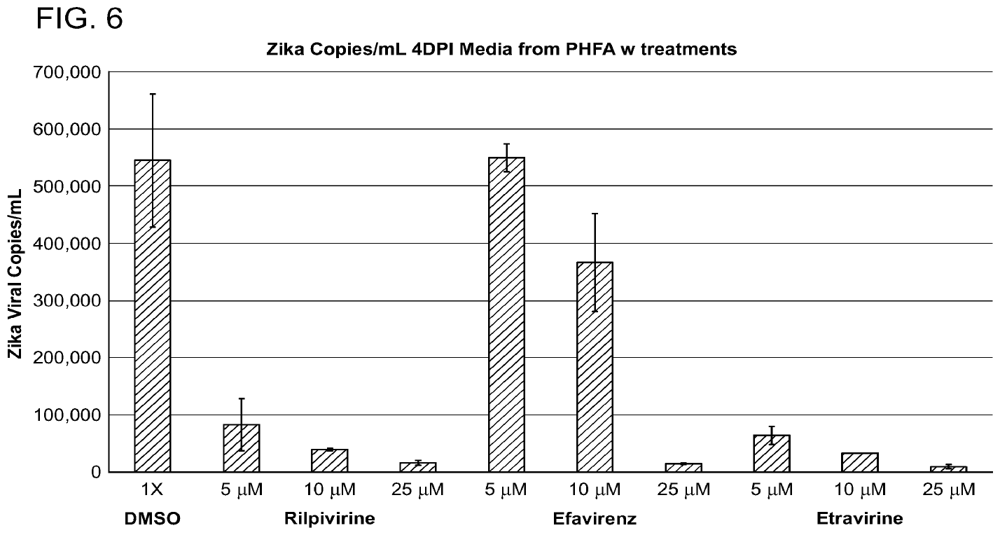

Several Non-Nucleoside (NNRTIs) and Nucleoside Reverse Transcriptase Inhibitors (NNRTIs) were analyzed and screened for the effect of these compounds on Zika virus propagation. Table 1 shows a list of anti-retroviral drugs tested for anti-ZIKV activities. The results are shown in FIGS. 1A, 1B-6. Interestingly, among the drugs used, TmC (Rilpivirine) showed a dramatic reduction in ZIKV copy numbers released by infected cells, a robust suppression in replicated viral genomic RNA, and a significant decrease in viral gene expressions.

These results provide evidence that ZIKV replication can be suppressed by the non-nucleoside reverse transcriptase inhibitor Rilpivirine.

TABLE 1
List of Antiretroviral drugs tested for anti-ZIKV activities.

| Antiretroviral drug | Effect |
|---|---|
| DDI (2′,3′ - Dideoxyinosine) | HIV-1 RT-inhibitor, Nucleoside analog |
| ddC (2′,3′ - Dideoxycytidine) | HIV-1 RT-inhibitor, Nucleoside analog |
| AZT (Zidovudine) | HIV-1 RT-inhibitor, Nucleoside analog |
| FTC (Emtricitabine) | HIV-1 RT-inhibitor, Nucleoside analog |
| TMC278 (Rilpivirine) | HIV-1 RT-inhibitor, Non Nucleoside RT |
| | inhibitor (NNRTI) |
| Nevi (Nevirapine) | HIV-1 RT-inhibitor, Non Nucleoside RT |
| | inhibitor (NNRTI) |
| ABA (Abacavir) | HIV-1 RT-inhibitor, Nucleoside analog |
| 3TC (Lamivudine) | HIV-1 RT-inhibitor, Nucleoside analog |
| EFA (Efavirenz) | HIV-1 RT-inhibitor, Non Nucleoside RT |
| | inhibitor (NNRTI) |
| ETRA (Etravirine) | HIV-1 RT-inhibitor, Non Nucleoside RT |
| | inhibitor (NNRTI) |

### Example 2: Rilpivirine Inhibits ZIKV in IFNR^{-/-} Mice

Materials and Methods

In order to test anti-ZIKV activity of rilpivirine, IFNR knockout (IFNR^{-/-}) mice were utilized. Two to four month old mice (male and female) were divided into three groups of three mice per group. Rilpivirine treatments were started for one group of mice at two days prior ZIKV infections. Rilpivirine concentration was 12.5 μg per 25 g mouse via intraperitoneal (IP) injections. Treatments were done daily. The second group of mice was treated with rilpivirine without ZIKV infection. The third group of mice were infected with ZIKV but not treated with rilpiverine. Mice were infected with the PRVABC59 strain of ZIKV (10,000 pfu in 20ul PBS) injected into the footpad on day 0. All mice were monitored daily with weight checks and grip test analysis measured every other day. Physical, behavior, and motor coordination/paralysis were also assessed at the time of the weight checks. Mice were euthanized based on weight loss, physical, behavior, and motor function deficits. All mice were euthanized at day 14 post infection.

Results

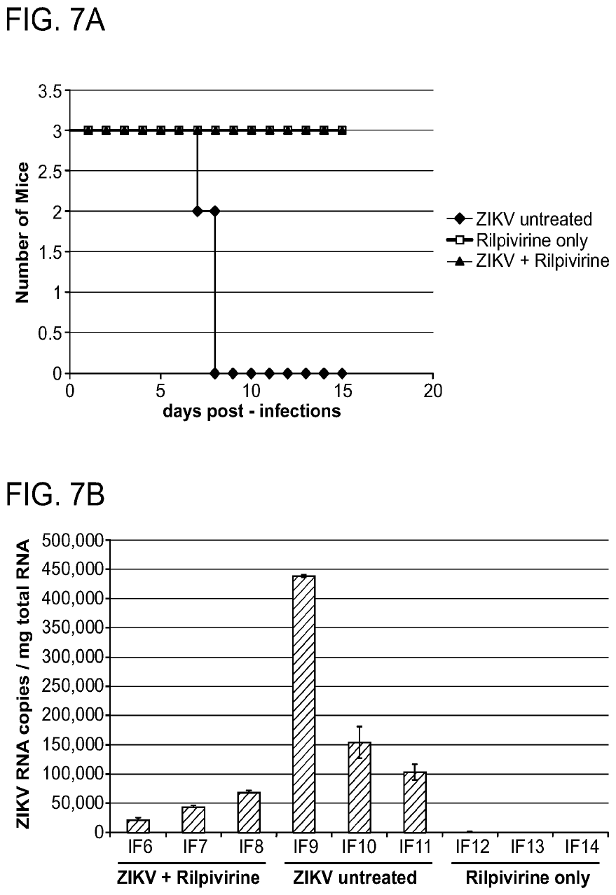

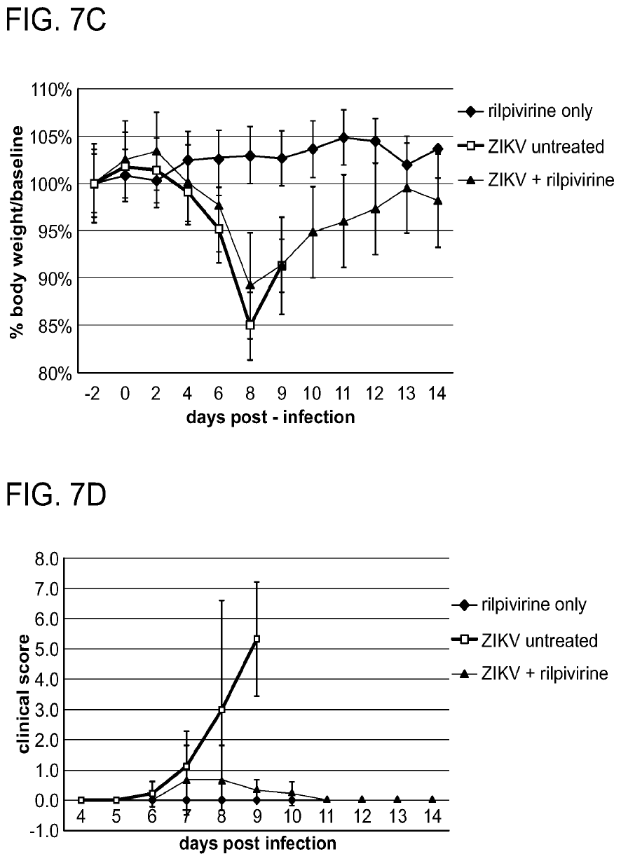

Kaplan-Meier estimates was performed to calculate survivor curves. As seen in FIG. 7A, one mouse was euthanized at 7 dpi and two others were euthanized at 8 dpi from the ZIKV infected (untreated) group providing evidence that ZIKV infection is highly lethal in IFNR^{-/-} mice. On the other hand, all mice from the ZIKV infected and rilpivirine treated groups survived providing evidence that rilpivirine was capable of suppressing ZIKV pathology in this animal model. ZIKV RNA copies were also analyzed in post-mortem brain tissues by Q-RT-PCR according to the protocol described by Lanciotti et al., (Emerg Infect Dis. 2008 August; 14(8):1232-9. doi: 10.3201/eid1408.080287). All real-time assays were performed by using the QUANTITECT Probe RT-PCR Kit (QIAGEN, Valencia, Calif., USA) with amplification in the iCycler instrument following the manufacturer's protocol. The following ZIKV specific primers were used in the reactions: ZIKV-1086 (1086-1102) CCGCTGCCCAACACAAG (SEQ ID NO: 1), ZIKV-1162c (1162-1139) CCACTAACGTTCTTTTGCAGACAT (SEQ ID NO: 2), ZIKV-1107-FAM (1107-1137) AGCCTACCTTGACAAGCAGTCAGACACTCAA-6FAM (SEQ ID NO: 3). As seen in FIG. 7B, ZIKV RNA copies in the brain were significantly reduced in mice treated with rilpivirine. Weight loss and grip test analyses presented in FIGS. 7C and 7D provided evidence and supported the anti-ZIKV activity of rilpivirine in the IFNR^{-/-} mice.

What is claimed:

1. A method of inhibiting a Flaviviridae virus replication in a cell, in vitro or in vivo comprising:
contacting a cell in vitro or administering to a subject, a therapeutically effective amount of a non-nucleoside reverse transcriptase inhibitor (NNRTI), analogs, variants or combinations thereof and/or a nucleoside reverse transcriptase inhibitor (NRTI), analogs, variants or combinations thereof, thereby
inhibiting Flaviviridae virus replication in a cell, in vitro or in vivo.

2. The method of claim 1, wherein an NNRTI comprises: etravirine, efavirenz, nevirapine,
rilpivirine, delavirdine, or nevirapine.

3. The method of claim 2, wherein one NNRTI or a combination of NNRTI's are administered to a subject.

4. The method of claim 2, wherein the NNRTI is rilpivirine.

5. The method of claim 1, wherein an NRTI comprises: lamivudine, zidovudine, emtricitabine, abacavir, zalcitabine, dideoxycytidine, azidothymidine, tenofovir disoproxil fumarate, didanosine (ddI EC), dideoxyinosine, stavudine, abacavir sulfate or combinations thereof.

6. The method of claim 4, wherein one or a combination of NRTI's are administered to a subject.

7. The method of anyone of claim 2 or 4, wherein a combination of one or more NNRTI's and one or more NRTI's are administered to a subject.

8. The method of claim 1, further comprising one or more protease inhibitors.

9. The method of claim 8, wherein a protease inhibitor comprises: amprenavir, tipranavir, indinavir, saquinavir mesylate, lopinavir and ritonavir (LPV/RTV), Fosamprenavir Calcium (FOS-APV), ritonavir, darunavir, atazanavir sulfate, nelfinavir mesylate or combinations thereof.

10. The method of claim 1, wherein the NNRTI's, NRTI's, analogs, variants or combinations thereof are administered as a pharmaceutical composition.

11. The method of claim 1, wherein the NNRTI's, NRTI's, analogs, variants or combinations thereof are administered to a subject in a delivery vehicle.

12. The method of claim 1, wherein an NNRTI, NRTI, analogs, variants or combinations thereof or combinations thereof are administered to a subject at high-risk of contracting a Flaviviridae virus infection.

13. The method of claim 1, wherein an NNRTI, NRTI, analogs, variants or combinations thereof are administered to a subject to prevent and/or treat a Flaviviridae virus infection.

14. The method of claim 1, wherein the Flaviviridae comprises: dengue virus, tick-borne encephalitis virus, West Nile virus, yellow fever virus, Japanese encephalitis virus, Kyasanur Forest disease virus, Alkhurma hemorrhagic fever virus, Omsk hemorrhagic fever virus, or Zika virus.

15. The method of claim 1, wherein the Flaviviridae virus is Zika virus.

16. The method of claim 1, optionally comprising administering one or more therapeutic agents.

17. The method of claim 16, wherein the one or more therapeutic agents comprise: antibiotics, anti-fungal, anti-inflammatory agents, anti-pyretics, chemotherapeutic agents, interferons, cytokines, monokines, antibodies, immunotherapeutics or combinations thereof.

18. A composition comprising a therapeutically effective amount of a non-nucleoside reverse transcriptase inhibitor (NNRTI), analogs, variants or combinations thereof and/or a nucleoside reverse transcriptase inhibitor (NRTI), analogs, variants or combinations thereof.

19. The composition of claim 18, wherein an NNRTI comprises: etravirine, efavirenz, nevirapine, rilpivirine, delavirdine, or nevirapine.

20. The composition of claim 18, wherein an NRTI comprises: lamivudine, zidovudine, emtricitabine, abacavir, zalcitabine, dideoxycytidine, azidothymidine, tenofovir disoproxil fumarate, didanosine (ddI EC), dideoxyinosine, stavudine, abacavir sulfate or combinations thereof.

21. The composition of claim 18, further comprising one or more protease inhibitors.

22. The composition of claim 21, wherein a protease inhibitor comprises: amprenavir, tipranavir, indinavir, saquinavir mesylate, lopinavir and ritonavir (LPV/RTV), Fosamprenavir Calcium (FOS-APV), ritonavir, darunavir, atazanavir sulfate, nelfinavir mesylate or combinations thereof.

23. The composition of claim 18, wherein the NNRTI's, NRTI's, analogs, variants or combinations thereof are comprised in a pharmaceutical composition.

24. The composition of claim 18, wherein the NNRTI's, NRTI's, analogs, variants or combinations thereof are comprised in a delivery vehicle.

25. The composition of claim 18, further comprising one or more therapeutic agents.

26. The composition of claim 25, wherein the one or more therapeutic agents comprise: antibiotics, anti-fungal, anti-inflammatory agents, anti-pyretics, chemotherapeutic agents, interferons, cytokines, monokines, antibodies, immunotherapeutics or combinations thereof.

27. A method of preventing and/or treating a subject at risk of contracting a Zika virus infection or infected with Zika virus, comprising:
administering to a subject, a therapeutically effective amount of a non-nucleoside reverse transcriptase inhibitor (NNRTI), analogs, variants or combinations thereof and/or a nucleoside reverse transcriptase inhibitor (NRTI), analogs, variants or combinations thereof; thereby,
preventing and/or treating a subject at risk of contracting a Zika virus infection or infected with Zika virus.

28. The method of claim 27, wherein an NNRTI comprises: etravirine, efavirenz, nevirapine,
rilpivirine, delavirdine, or nevirapine.

29. The method of claim 27, wherein one NNRTI or a combination of two or more NNRTI's are administered to a subject.

30. The method of claim 27, wherein the NNRTI is rilpivirine.

31. The method of claim 30, wherein an NRTI comprises: lamivudine, zidovudine, emtricitabine, abacavir, zalcitabine, dideoxycytidine, azidothymidine, tenofovir disoproxil fumarate, didanosine (ddI EC), dideoxyinosine, stavudine, abacavir sulfate or combinations thereof.

32. The method of claim 27, wherein one or a combination of two or more NRTI's are administered to a subject.

33. The method of claim 27, wherein a combination of one or more NNRTI's and one or more NRTI's are administered to a subject.

34. The method of claim 27, further comprising one or more protease inhibitors.

35. The method of claim 34, wherein a protease inhibitor comprises: amprenavir, tipranavir, indinavir, saquinavir mesylate, lopinavir and ritonavir (LPV/RTV), Fosamprenavir Calcium (FOS-APV), ritonavir, darunavir, atazanavir sulfate, nelfinavir mesylate or combinations thereof.

36. The method of claim 27, wherein the NNRTI's, NRTI's, analogs, variants or combinations thereof are administered as a pharmaceutical composition.

37. The method of claim 27, wherein the NNRTI's, NRTI's, analogs, variants or combinations thereof are administered to a subject in a delivery vehicle.

38. A composition comprising a therapeutically effective amount of a non-nucleoside reverse transcriptase inhibitor (NNRTI), analogs, variants or combinations thereof and/or a nucleoside reverse transcriptase inhibitor (NRTI), analogs, variants or combinations thereof and one or more agents.

39. The composition of claim 38, wherein the one or more agents comprise: antibiotics, anti-fungal, anti-inflammatory agents, anti-pyretics, chemotherapeutic agents, interferons, cytokines, monokines, antibodies, immunotherapeutics or combinations thereof.
